(19) United States
(12) Patent Application Publication
(10) Pub. No.: US 2009/0307970 A1
(43) Pub. Date: Dec. 17, 2009

(54) METHOD OF GROWING PLANTS

(76) Inventor: Edwin Henricus Antonius HOLMAN, Ootmarsum (NL)

Correspondence Address: BANNER & WITCOFF, LTD., 28 STATE STREET, SUITE 1800, BOSTON, MA, 02109-1701, US

(21) Appl. No.: 12/139,753

(22) Filed: Jun. 16, 2008

(51) Int. Cl. A01N 27/00; A01H 5/00; A01P 21/00; A01G 9/00

(52) U.S. Cl. 47/17; 504/357; 800/298

(57) ABSTRACT

The invention relates to a method of growing plants, comprising the step of allowing a seedling, tissue culture or plantlet to develop into a plant, wherein during early ontogeny of said plant an increase in the rate of respiration and/or protein turnover due to oxidative stress is prevented.

### FIELD

The present invention is in the field of crop production. More in particular, the present invention relates to methods for growing plants and methods for increasing the average yearly crop production of a crop production area. The invention further relates to transgenic plants transformed with a gene encoding the enzyme isoprene synthase, and to greenhouses comprising means for controlling the atmospheric content of a reactive oxygen species.

### BACKGROUND

Global demand for wheat, rice, corn, and other essential grains is expected to steadily rise over the next twenty years. Apart from serving as a food source, the demands will rise as plant-based fuel and chemicals production by biological processes is growing. Meeting this demand by increasing production through increased land use is not very likely; and while better crop management may make a marginal difference, most agriculture experts agree that this anticipated deficit must be made up through increased crop yields.

Modern crop production management systems are tailored to optimize each and every parameter that influences crop yield. Management of moisture and nutrient availability and control of pests is standard in all agricultural production schemes for field crops. In greenhouse crops, availability of CO₂ and light and temperature may in addition be optimized. Hence, most crops are currently produced under apparently optimal conditions. Although some variation is perceived as “natural,” realistic production efficiencies are still a fraction of theoretical maximum levels. In fact, year-average efficiency levels attained in the field for most crops are often less than half of the maximum levels observed in particular years. The reason for this difference is not properly understood. Hence, there is a need for further understanding and optimizing biological production efficiencies.

Plant biomass production is determined by the plants net carbon fixation efficiency. This efficiency is governed by the rate of carbon gain in terms of CO₂ fixation by photosynthesis and the rate of carbon loss in terms of CO₂ emission by respiration. While gross photosynthesis rises with temperature, so does respiration and whereas the photosynthesis rate tends to flatten at the optimum of the photosynthetic enzyme rubisco of about 25° C., respiration continues to rise rapidly above this temperature and roughly doubles every 10° C. (Q₁₀ is usually 2). At temperatures above about 35° C. all the sugar produced is used to support respiration. At temperatures higher than 35° C., plants respire more sugar than they can produce which leads to deterioration and ultimate death of the plant. Consequently the net photosynthesis (the production of energy compounds minus their use by respiration including photorespiration) must be considered when attempting to optimize production. As noted above, traditional methods for improving carbon balance are aimed at improving the gross photosynthesis rate. Although a relationship between respiration rate and temperature is well known, and respiration is known to vary depending on protein turnover and maintenance requirements, baseline respiration rates are generally believed to be relatively fixed.

During their life cycle, plants undergo a large number of physiological, biochemical and morphological changes that are controlled by alterations in gene expression. Yet, the morphogenetic capacity of plants is a function of both genetic and environmental parameters, of which thermal and light conditions appear to have the greatest influence. Plant ontogeny, the sum of morphogenic processes that describe the development of a plant from seed germination through to maturity, is known to influence plant production parameters, such as the degree to which plants compensate after defoliation or herbivore damage. Also, it is known that factors that affect crop growth early in ontogeny often produce modifications that extend through the season and may be manifest in altered economic yield. It is believed that this is the result of epigenetic factors, among which DNA methylation is one of the best know. Plant ontogeny includes developmental changes in plant architecture, storage capacity, and resource allocation to different functions (e.g., storage, defense, reproduction). In the case of woody species, an increase in the plant age is associated with changes in resource allocation patterns, as the carbon/nutrient balance, storage capacity, and access to water and nutrients usually increase, while root to shoot ratio, growth rate, photosynthesis, stomatal conductance, hormone production, and metabolic activity typically decrease. In addition, morphological differences between juvenile and adult trees include variation in leaf morphology, phyllotaxy, shoot orientation, seasonal leaf retention, presence of adventitious roots, and leaf-specific mass.

It is however not known whether crop production in general is influenced ontogenic processes, or whether factors that affect crops early in ontogeny can produce modifications that extend throughout the life of the plant.

### SUMMARY

The present inventor has now discovered that baseline respiration rates are fixed early in the ontogeny of plants. In fact, the inventor has discovered that oxidative stress early in the ontogeny of plants results in plants having a high baseline respiration rate for the rest of their productive life span. Although in mature plants, the respiration rate may temporarily increase in response to altered environmental conditions, it will return to a rate the level of which is ultimately fixed at an early stage of development.

Without wishing to be bound by any theory, it is hypothesized that early in ontogeny plants determine a default respiration rate based on the conditions that prevail at that time in their life. An increase in protein turnover, for instance due to increased damage, will result in a higher respiration rate. It is further hypothesized that this is essentially a one-way street: the baseline respiration rate can go up, but it cannot come down. As a result, a plant that is exposed to unfavorable conditions such as high ozone concentrations that result in leaf damage and increased respiration rates early in ontogeny, will keep high baseline respiration rates essentially throughout its life, or at least for prolonged periods of time. Hence, such a plant will exhibit low net production rates.

This amazing discovery was done in Brazil, where annual sugar cane burnings result in high atmospheric concentrations of ozone. It was found that plants that were planted in a period wherein ozone concentrations were lowest consistently exhibited high production, whereas plants that were planted in periods that coincided with early ontogenic exposure to these adverse conditions consistently exhibited low production rates.

In a first aspect, the present invention now provides a method of growing plants, comprising the step of allowing a seedling, tissue culture or plantlet to develop into a plant, wherein during early ontogeny of said plant an undesirable increase in the rate of respiration and/or protein turnover due to oxidative stress is prevented.

In a preferred embodiment of this method, the oxidative stress is the result of damage to said plant brought about by radiation or reactive oxygen species, preferably H₂O₂ or O₃.

In another preferred embodiment of the method of the invention, the oxidative stress is prevented by controlling the content of a reactive oxygen species in the growth environment. Said reactive oxygen species in the growth environment is preferably O₃ in the growth atmosphere. The O₃ content may be controlled in one of many ways. For instance in a closed system, it may be controlled by using O₃ scrubbers. A suitable O₃ scrubber is for instance an activated carbon filter. Alternatively, the O₃ content may be controlled by exposing the plant or its growth atmosphere to isoprene. Isoprene is known to neutralize ozone. In a further preferred embodiment, the isoprene may be endogenously produced in leaves of a transgenic or recombinant plant transformed with a gene encoding the enzyme isoprene synthase.

In yet another preferred embodiment of the method of the invention, the early ontogeny of said plant is the prefloral stage, preferably a period from 1-6 months post-germination.

In yet another preferred embodiment of the method of the invention, the plant is a perennial plant or a crop plant.

In another aspect, the present invention provides a method for increasing the average yearly crop production of a crop production area, comprising growing a plant according to a method of the invention as described above and using said plant as a crop plant in said production area.

In a preferred embodiment of this method, the increase in the average yearly crop production is brought about by an improved net carbon fixation efficiency, biomass production, dry matter content and/or pest resistance of said crop plant.

In another aspect, the present invention provides a transgenic plant transformed with a gene encoding the enzyme isoprene synthase. Preferably, the transgenic plant is grown by the method of the present invention as described above, wherein during early ontogeny of said plant an undesirable increase in the rate of respiration and/or protein turnover due to oxidative stress is prevented.

In yet another aspect, the present invention provides a plant part obtained from a plant of the present invention as described above.

In still a further aspect, the present invention provides a greenhouse for growing plants comprising an atmosphere wherein the content of a reactive oxygen species is controlled. Preferably, said reactive oxygen species is O₃ and the greenhouse of the present invention is therefore preferably provided with O₃ scrubbers.

In yet another aspect, the present invention provides a method for optimizing the production of a specific type of plant on a selected cultivated area, comprising the steps of:

a) determining for said specific type of plant the early ontogenic phase wherein the baseline respiration rate is fixed to a permanent minimum level;

b) monitoring in the air over said cultivated area the concentration of reactive oxygen species, for instance by using an atmospheric sensor capable of determining the concentration of reactive oxygen species in air; and

c) sewing a seed or planting a seedling or plantlet of said specific type of plant and allowing the seedling or plantlet to develop into a plant, wherein during said early ontogenic phase of said plant an exposure to undesirable concentrations of reactive oxygen species from the air over said cultivated area is essentially prevented by selecting the moment of sewing or planting using the data obtained in step b).

### DETAILED DESCRIPTION

The term “plant,” as used herein, refers to any type of plant. The inventors have provided below an exemplary description of some plants that may be used with the invention. However, the list is provided for illustrative purposes only and is not limiting, as other types of plants will be known to those of skill in the art and could be used with the invention.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet and fodder beet), sweet potatoes, swiss-chard, horseradish, tomatoes, kale, turnips, and spices.

Other types of plants frequently finding commercial use include fruit and vine crops such as apples, apricots, cherries, nectarines, peaches, pears, plums, prunes, quince almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pornes, melon, mango, papaya, and lychee.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts), fiber plants (cotton, flax, hemp, jute), lauraceae (cinnamon, camphor), or plants such as coffee, sugarcane, tea, and natural rubber plants.

Another economically important group of plants are ornamental plants. Examples of commonly grown ornamental plants include alstroemeria (e.g., Alstoemeria brasiliensis), aster, azalea (e.g., Rhododendron sp.), begonias (e.g., Begonia sp.), bellflower, bouganvillea, cactus (e.g., Cactaceae schlumbergera truncata), camellia, carnation (e.g., Dianthus caryophyllus), chrysanthemums (e.g., Chrysanthemum sp.), clematis (e.g., Clematis sp.), cockscomb, columbine, cyclamen (e.g., Cyclamen sp.), daffodils (e.g., Narcissus sp.), false cypress, freesia (e.g., Freesia refracta), geraniums, gerberas, gladiolus (e.g., Gladiolus sp.), holly, hybiscus (e.g., Hibiscus rosasanensis), hydrangea (e.g., Macrophylla hydrangea), juniper, lilies (e.g., Lilium sp.), magnolia, miniroses, orchids (e.g., members of the family Orchidaceae), petunias (e.g., Petunia hybrida), poinsettia (e.g., Euphorbia pulcherima), primroses, rhododendron, roses (e.g., Rosa sp.), snapdragons (e.g., Antirrhinum sp.), shrubs, trees such as forest (broad-leaved trees and evergreens, such as conifers) and tulips (e.g., Tulipa sp.).

The term “plant part,” as used herein, includes reference to, but is not limited to, single cells and tissues from microspores, pollen, ovules, leaves, embryos, roots, root tips, anthers, flowers, fruits, seeds, stems, shoots, scions, rootstocks, protoplasts, calli, meristemic tissues and the like.

The term “perennial,” as used herein, refers to a plant that lives for more than two years. Perennial plants can be short-lived (only a few years) or they can be long-lived, as some woody plants, such as trees.

The term “crop plant,” as used herein, refers to a plant which is harvested or provides a harvestable product.

The terms “seedling” and “plantlet,” as used herein, are interchangeable and refer to the juvenile plant grown from a sprout, embryo or a germinating seed and generally includes any small plant showing well developed green cotyledons and root elongation and which is propagated prior to transplanting in the ultimate location wherein it is to mature.

The term “tissue culture,” as used herein, refers to a culture of plant cells wherein the cells are propagated in a nutrient medium under controlled conditions.

The term “early ontogeny,” as used herein, refers to the early phase in the course of growth and development of an individual to maturity. This phase will differ in length between plant species. For roses, this period is estimated to amount to about 6 to 8 months. In essence, the early ontogenetic phase terminates when the plant characteristics in response to environmental stimuli, such as respiration rate, become established and fixed. The skilled person can determine the early ontogenetic phase for any plant species by measuring the ozone-induced respiration rate in plants at periodic intervals and determining at what age of the plant the increase in respiration rate due to ozone exposure does not return to pre-exposure values, but is fixed.

The term “epigenetic,” as used herein, refers to the state of the DNA with respect to heritable changes in function without a change in the nucleotide sequence. Epigenetic changes can be caused by modification of the DNA, in particular chromatin remodeling caused by modifications of the histone proteins and DNA methylation. These changes affect gene transcription and ultimately affect phenotype. Epigenetic changes thus involve factors that influence behavior of a cell without directly affecting its DNA or other genetic components. The epigenetic view of differentiation is that cells undergo differentiation events that depend on correct temporal and spatial repression, derepression, or activation of genes affecting the fate of cells, tissues, organs, and ultimately, organisms. Thus epigenetic changes in an organism are normal and result in alterations in gene expression. For example, epigenetic transformation of a normal cell to a tumor cell can occur without mutation of any gene.

The term “respiration,” as used herein, refers to the process by which O₂ is transported to and used by the cells and CO₂ is produced and eliminated from the cells during which process organic matter is oxidized.

The term “protein turnover,” as used herein, refers to the flow of amino acids from existing protein into newly synthesized protein. Protein turnover is generally regarded as one of the most important maintenance processes in plants in terms of energy requirements. Both biosynthetic and breakdown processes affect the rate of protein turnover. Both protein synthesis and protein degradation require respiratory energy. Protein turnover has several important functions in regulating the plant's metabolism. Together with protein synthesis, degradation is essential to maintain appropriate enzyme levels and to modulate these levels based on internal and external signals. Furthermore, protein degradation is important in allowing a plant to cope with changing environmental conditions. When nutrients become limiting, the rate of protein turnover is accelerated by increasing the rate of degradation relative to synthesis, which generates a pool of free amino acids from less essential proteins that can be used to assemble more essential ones.

The term “oxidative stress,” as used herein, refers to the state in which cells are exposed to excessive levels of molecular oxygen or reactive oxygen species (ROS) to the extent that damage is incurred and cellular repairs systems are mobilized. Oxidative stress may be measured by increased protein turnover rates.

The term “radiation,” as used herein, includes both particle radiation (i.e., electrons, protons), high-energy electromagnetic radiation (i.e., x-rays, gamma rays) and other ionizing radiation in the radiant-energy spectrum, as well as non-ionizing electromagnetic radiation, and radiation in the ultraviolet, visible light, and infra-red spectrum.

The term “reactive oxygen species” (ROS), as used herein, refers to oxygen ions, free radicals, and peroxides, both inorganic and organic. They are generally very small molecules and are highly reactive due to the presence of unpaired valence shell electrons. ROSs form as a natural byproduct of the normal metabolism of oxygen and have important roles in cell signaling. However, during times of environmental stress ROS levels can increase dramatically, which can result in significant damage to cell structures. This cumulates into a situation known as oxidative stress.

The term “growth environment,” as used herein, refers to the soil, substrate or air in which the plant is growing.

The term “scrubber,” as used herein with reference to reactive oxygen species or O₃ (ozone) scrubber, refers to a system, device, or chemical capable of binding or chemically converting reactive oxygen species, such as O₃ to the effect that said reactive oxygen species is eliminated from the environment which is placed in contact with the scrubber.

The term “isoprene,” as used herein, refers to the chemical compound 2-methylbuta-1,3-diene.

The term “endogenous” as in “endogenously produced” refers to produced within the plant (cell).

The terms “transgenic” and “recombinant,” as used herein, refer to a plant comprising a heterologous or homologous gene from another species, introduced therein by genetic engineering or recombination.

The term “transformed” as used herein refers to a process for making or introducing a stable change in the characteristics (express phenotype) of a cell by the mechanism of gene transfer whereby DNA or RNA is introduced into a cell in a form where it expresses a specific gene product or alters an expression or affects endogenous gene products. The vector can be introduced into the cell by a variety of methods including microinjection, CaPO4 precipitation, lipofection (liposome fusion), use of a gene gun and DNA vector transporter.

The term “vector” as used herein refers to a construction comprised of genetic material designed to direct transformation of a targeted cell. A vector contains multiple genetic elements positionally and sequentially oriented, i.e., operatively linked with other necessary elements such that the nucleic acid in a nucleic acid cassette can be transcribed and when necessary, translated in the transformed cells.

The term “gene,” as used herein, refers to a DNA coding region flanked by 5′ and/or 3′ regulatory sequences allowing an RNA to be transcribed which can be translated to a protein, typically comprising at least a promoter region.

The term “isoprene synthase,” as used herein, refers to the enzyme with registry number EC 2.5.1.-, that catalyses the elimination of pyrophosphate from dimethylallyl diphosphate to form isoprene. The amino acid sequence of the enzyme in Populus alba is provided in SEQ ID NO:1. The term is considered to cover homologues having >80%, preferably >90% sequence identity with SEQ ID NO:1.

The term “prefloral stage,” as used herein, refers to the ontogenic stage preceding the emergence of reproductive structures in a plant.

The term “post-germination,” as used herein, refers to the period in the development of the plant following the emergence of the radicle from the seed.

The term “average yearly crop production,” as used herein, refers to the amount of crop produced per annum or season (average seasonal crop production) in the form of weights or number of plants or plant parts harvested or weight gain in crop biomass are expressed per unit of production area, and wherein the individual amounts per annum for multiple years are summated and divided by the number of years.

The term “production area,” as used herein, refers to a location where plants are grown and where products in the form of plants or plant parts are produced for harvest. The size of the production area is generally expressed in square meters or acres of land. A production area can be an open field or a greenhouse.

The term “net carbon fixation efficiency,” as used herein, is used interchangeable with the term “net photosynthetic efficiency” and refers to the net efficiency with which carbon dioxide is converted into organic compounds, taking into account the losses due to respiration.

The term “biomass production,” as used herein, refers to the production of plant derived organic material.

The term “dry matter content,” as used herein, refers to the mass fraction (%) that remains after the water fraction (%) has been removed by drying.

The term “pest resistance,” as used herein, refers to resistance against viral, bacterial, fungal, and insect pests, as well as pests by molluscs and nematodes.

The term “greenhouse,” as used herein, refers to any structure comprising walls, a roof, and a floor designed and used principally for growing plants in a controlled and protected environment. The walls and roof are usually constructed of transparent or translucent material to allow passage of sunlight for plant growth.

The methods of growing plants according to the present invention is based on three essential realizations:
i) respiration rates reached in a plant during early ontogeny culminate in the development of a baseline respiration rate, which is the minimum rate at which said plant will respire when mature;
ii) this baseline respiration rate is attained by one or more step-up increments and is ultimately fixed at baseline level by epigenetic changes; and
iii) the step-up increments in the respiration rate are the plant's response to increased protein turnover rates which in turn are the result of oxidative stress experienced by said plant.

Therefore, when attempting to keep the respiration rates in mature plants as low as possible, it is important to minimize the oxidative stress to said plant during the phase wherein the baseline respiration rate is fixed by epigenetic factors, that is, early in ontogeny.

Hence, a method of growing plants according to the present invention comprises the important step of allowing a seedling, tissue culture or plantlet to develop into a plant and to pass through its early ontogenic phase wherein epigenetic factors fix the baseline respiration rate to a permanent minimum level. The early ontogenic phase wherein epigenetic factors fix the baseline respiration rate to a permanent level can differ for different plants, and can therefore not be defined for each and every plant species in advance. Also, the phase at which epigenetic factors determine the permanent minimum rate of respiration may vary between plant varieties within a species. This phase may however be experimentally determined for each and every plant species or plant variety as follows:

Seedlings, tissue cultures or plantlets are allowed to develop into mature plants. The total development time is recorded and divided into a large but practical number of regular intervals (between 2 and for instance 10, 20, 50, 100, or 1000). During each interval, the respiration rate of the developing plant is measured using standard techniques. After each measurement, the plant is temporarily subjected to oxidative stress, for instance by exposing it to ozone. The oxidative stress can optionally be applied at incrementally increasing levels during the different intervals. The early ontogenic phase wherein epigenetic factors fix the baseline respiration rate to a permanent level is now determined from the data obtained as:

a) the total number of intervals preceding the interval in which the respiration rate returns to a baseline level after said oxidative stress period, or

b) the total number of intervals during which the respiration rate shows a step-wise increase relative to the preceding interval and no decrease in the subsequent or following interval.

Roughly, in a method of the present invention the early ontogenic phase wherein epigenetic factors fix the baseline respiration rate to a permanent level is the prefloral stage, preferably a period from 1-6 months post-germination.

A method of growing plants according to the present invention further comprises the important step of preventing the during early ontogeny of said plant the occurrence of an undesirable increase in the rate of respiration and/or protein turnover due to oxidative stress. It is to be understood that in accordance with the above-described model for epigenetic fixation of baseline respiration rates, an undesirable increase in the rate of respiration is equivalent to an undesirable increase in the rate of protein turnover. Hence, preventing the one from occurring automatically also assures prevention of the other to occur.

Oxidative stress is often, but not exclusively, the result of damage to said plant brought about by radiation or reactive oxygen species. Hence, the prevention of an undesirable increase in the rate of respiration and/or protein turnover due to oxidative stress can be attained by preventing exposure to radiation or reactive oxygen species that result in such an increase. Most notably, intracellular H₂O₂ levels and atmospheric O₃ concentrations are maintained at sub-stress levels. Such levels may differ between plant species and between plant varieties, but can be easily determined experimentally. For instance, a plant can be exposed to a certain test level of radiation or reactive oxygen species for a predetermined period of time (i.e. varying between several minutes to several weeks) and the rate of respiration and/or protein turnover is determine before and after said exposure. A sub-stress level of radiation or reactive oxygen species is the level at which the exposure does not result in an increase of the post-exposure rate relative to the rate before.

Thus, in preferred embodiments of methods of the present invention, the oxidative stress is prevented by controlling the content of a reactive oxygen species in the growth environment such that they remain at sub-stress levels.

The growth environment may be the soil, substrate, medium or atmosphere in which the plant is grown. Preferably the content of the reactive oxygen species, most preferably O₃, is controlled in the growth atmosphere.

In order to prevent oxidative stress, the O₃-content of the growth atmosphere of the plant during the early ontogenetic phase is preferably kept below 100 ppbv, most preferably below 75 ppbv, even more preferably below 60 ppbv, still more preferably below 50 ppbv, even more preferably below 40 ppbv, yet even more preferably below 35, 30, 25, 20, 15, 10, 5 or 1 ppbv, wherein ppbv refers to parts per billion by volume. Sporadic increases are likely not to have a major effect on the plants, yet values above 100 ppbv should essentially be avoided at all times during the early ontogenetic phase.

Essentially, this may be attained in different ways. For instance, the content in the total airspace (atmosphere) in which the plant is grown may be controlled. Control in the total airspace will generally involve the use of closed cultivation systems, such as greenhouses. Effective control of the level of reactive oxygen species can be obtained by the use of scrubbers or air filtering systems. Such filtering systems are well known in the field of conditioning of air. Suitable scrubbers or filter materials include those materials that absorb or adsorb reactive oxygen species. An example of a suitable material is activated carbon, but other materials capable of filtering reactive oxygen species from air that is passed through the filter of over the filter material are also suitable. It is preferred that an air circulation system is used wherein the air is moved over or through the scrubber. Also suitable is the use of a material that reacts with the reactive oxygen species in the atmosphere, thereby rendering the reactive oxygen species unreactive. A suitable example of such a material is isoprene. Hence, a greenhouse may periodically be loaded with isoprene gas in order to remove reactive oxygen species from the air. Generally, an amount of isoprene equivalent to 1-1000, more preferably 50-150 parts per billion by volume (ppbv) of air is sufficient.

Alternatively, only the content in the air that is in direct contact with the plant surface may be controlled. This is most effectively attained by providing the plant with an endogenous source of isoprene. To this end, isoprene may be endogenously produced in leaves of a plant. In plants that do not contain in their genome a (functional) biosynthetic pathway for the production of isoprene the missing genes for such a pathway may be introduced by providing a transgenic or recombinant plant transformed with said genes. One of said genes is the gene encoding the enzyme isoprene synthase. The skilled person is well aware of the various methods and techniques for producing such transgenic plants. Particular reference is made to Sharkey et al. Plant Physiol., 2005 February; 137(2):700-712, which publication describes in detail the transformation of Arabidopsis (Arabidopsis thaliana) with the genomic isoprene synthase gene from kudzu (Pueraria Montana). This disclosure is incorporated herein in its entirety by reference.

A method of growing a plant according to the present invention has as an advantage that the net biosynthetic efficiency of said plant is increased during its entire lifetime. The invention is therefore especially important for plants that have a life cycle of multiple years, because it is in these plants that a high baseline respiration rate attained during early ontogeny has the most impact on the total production of the plant over its entire lifetime. Hence a plant in aspects of the present invention is preferably a perennial plant.

It will also be appreciated that the methods of the present invention are particularly suitable for plants in which production parameters are very important, such as for instance in crop plants.

A method of growing a plant according to the present invention is preferably practices on a large number of plants simultaneously as a part of crop production. Hence, in another aspect, the present invention provides a method for increasing the average yearly crop production of a crop production area, comprising growing a plant according to a method of the invention as described above and using said plant as a crop plant in said production area.

It is an advantage of the method of the present invention that the average yearly crop production is no longer adversely affected by seasonal fluctuations in the atmospheric load of reactive oxygen species.

A crop production area can be a small field of a few square meters, but most preferably is a large production area covering several acres or even a large number of square miles. The advantages of the present invention are best appreciated when a large number of plants are involved. The methods of the present invention can be performed on large cultivated areas, and can for instance comprise the monitoring of environmental factors that influence oxidative stress, and selecting the planting or sewing season such that in tits early ontogenic phase as defined herein the plant is exposed to a minimum of oxidative stress. Thus, the method of the invention can be made part of a crop cultivation system comprising the monitoring of for instance atmospheric reactive oxygen species, and using this information to select the optimal planting or sewing season for the crop such that the crops are not exposed to undesirable or stress-level concentrations of atmospheric reactive oxygen species such as O₃.

Based on the teachings of the present invention, the skilled person will understand that certain planting seasons in Brazil are best avoided due to excessive atmospheric concentrations of reactive oxygen species during sugar cane burning. It is an aspect of the present invention that such avoidance can be managed effectively by monitoring the concentration of reactive oxygen species in the air over a cultivated area using atmospheric pollution sensors or “sniffers” for determining the concentration of reactive oxygen species in air.

Therefore in another aspect, the present invention provides a method for optimizing the production of a specific type of plant on a selected cultivated area, comprising the steps of:

a) determining for said specific type of plant the early ontogenic phase wherein epigenetic factors fix the baseline respiration rate to a permanent level, for instance by using methods as described above;

b) monitoring for said selected cultivated area the concentration of reactive oxygen species in the air over said cultivated area, for instance by using an atmospheric sensor capable of determining the concentration of reactive oxygen species in air, or sampling air and determining the concentration in said sample; and

c) sewing a seed or planting a seedling or plantlet of said specific type of plant and allowing the seedling or plantlet to develop into a plant, wherein during early ontogeny of said plant an increase in the rate of respiration and/or protein turnover due to oxidative stress as a result of reactive oxygen species in the growth atmosphere is essentially prevented, by selecting the moment of sewing or planting using the data obtained in step b).

It should be understood that the monitoring in step b) need not be constant but must be frequent enough to allow the determination of the optimal moment for sewing or planting. The optimal moment for sewing or planting is the moment that assures that the subsequent early ontogenetic phase wherein the baseline respiration rate is fixed to its minimum level coincides with the lowest concentrations of reactive oxygen species in the growth atmosphere.

When reference is made herein to terms such as optimizing the production or increase in the average yearly crop production, it is meant that such attributes include or are brought about by an improved net carbon fixation efficiency, biomass production, dry matter content and/or pest resistance of said crop plant.

The invention further provides a transgenic plant transformed with one or more genes in of the biosynthetic pathway for the production of isoprene. Such genes include genes encoding the enzyme isoprene synthase. Isoprene synthase converts dimethylallyl diphosphate, derived from the methylerythritol 4-phosphate (MEP) pathway, to isoprene. Isoprene synthase genes of kudzu and aspen (Populus tremuloides) may for instance be used in aspects of the present invention. The skilled person is well aware of the methods by which such a transgenic plant can be produced (vide supra). The transgenic plant is preferably an plant exploited in agriculture such as a plant of a vegetable crop, a fruit or vine crop, or a field crop, or an ornamental plant. It is an aspect of the invention that transgenic Arabidopsis thaliana is expressly disclaimed herein and not a part of the present invention.

Preferably, the transgenic plant is grown by the method of the present invention as described above, wherein during early ontogeny of said plant an undesirable increase in the rate of respiration and/or protein turnover due to oxidative stress is prevented.

Another aspect of the present invention is a greenhouse for growing plants, in particular by methods of the present invention. The greenhouse of the present invention comprises means for controlling the content of a reactive oxygen species in the greenhouse atmosphere. Preferably, said greenhouse is equipped with a scrubber for removing reactive oxygen species from the growth environment. In addition, the greenhouse may be equipped with systems or devices for monitoring the concentration of reactive oxygen species in the greenhouse atmosphere (i.e., the air inside the greenhouse), for instance in the form atmospheric sensors capable of determining the concentration of reactive oxygen species in air.

### EXAMPLE

As an example, the present invention may be performed as follows. In a greenhouse roses are grown under normal conditions of temperature, water, nutrients and light. In addition, the greenhouse atmosphere is controlled for the amount of ozone, for instance by using an ozone scrubber in order to reduce the amount of ozone in the greenhouse atmosphere. Furthermore, in order to avoid entry of ozone from the outside atmosphere, the greenhouse is well closed.

At least for the duration of the early ontogenetic period as defined herein above, the ozone levels in the greenhouse are maintained at the lowest possible levels, preferably below 20-30 ppbv. Thereafter, that is after the early ontogenetic phase, the plants may be grown under less stringent conditions or the plants may be brought outside the greenhouse for further growth.

The exposure to the beneficial growth environment will result in plants having a lower respiration rate when mature—compared to plants being exposed to high ozone environments, and such plants will exhibit higher production output as described herein above.

1. A method of growing plants, comprising:
developing a seedling, tissue culture or plantlet into a plant; and
reducing or preventing an increase in rate of respiration or protein turnover resulting from oxidative stress during early ontogeny of the plant.

2. The method according to claim 1, wherein said oxidative stress is brought about by radiation or reactive oxygen species.

3. The method according to claim 1, wherein content of a reactive oxygen species is controlled in the growth environment.

4. The method according to claim 3, wherein said reactive oxygen species in the growth environment is O₃ or H₂O₂ in the growth atmosphere.

5. The method according to claim 4, wherein said O₃ content is controlled by using O₃ scrubbers.

6. The method according to claim 4, wherein said O₃ content is controlled by exposing said plant or its growth atmosphere to isoprene.

7. The method according to claim 6, wherein said isoprene is endogenously produced in leaves of a transgenic or recombinant plant transformed with a gene encoding the enzyme isoprene synthase.

8. The method according to claim 1, wherein the early ontogeny of said plant is the prefloral stage.

9. The method according to claim 1, wherein said plant is a perennial plant or a crop plant.

10. The method for increasing the average yearly crop production of a crop production area, comprising growing a plant according to the method of claim 1 and using said plant as a crop plant in said production area.

11. The method according to claim 10, wherein said increase in the average yearly crop production is brought about by an improved net carbon fixation efficiency, biomass production, dry matter content and/or pest resistance of said crop plant.

12. A transgenic plant transformed with a gene encoding the enzyme isoprene synthase.

13. A transgenic plant transformed with a gene encoding the enzyme isoprene synthase, wherein said plant is grown by the method of claim 1.

14. A plant part obtained from a plant according to claim 12.

15. A greenhouse for growing plants comprising an atmosphere wherein the content of a reactive oxygen species is controlled.

16. The greenhouse according to claim 15, wherein said reactive oxygen species is O₃.

17. The greenhouse according to claim 15, provided with O₃ scrubbers.

18. A method for optimizing the production of a plant in a cultivated area, comprising the steps of:
a) determining for the plant the early ontogenic phase wherein baseline respiration rate is fixed to a permanent minimum level;
b) monitoring in air over said cultivated area the concentration of reactive oxygen species; and
c) sewing a seed or planting a seedling or plantlet of said plant and allowing the seedling or plantlet to develop into a plant, wherein during said early ontogenic phase of said plant an exposure to undesirable concentrations of reactive oxygen species from the air over said cultivated area is reduced or prevented.


SEQUENCE LISTING (1 sequence)

SEQ ID NO: 1 - protein, 595 aa, Populus alba
MATELLCLHRPISLTHKLFRNPLPKVIQATPLTLKLRCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER